(19) United States
(12) Patent Application Publication
(10) Pub. No.: US 2019/0343489 A1
(43) Pub. Date: Nov. 14, 2019

(54) ULTRASOUND DIAGNOSIS APPARATUS AND MEDICAL INFORMATION PROCESSING METHOD

(71) Applicant: CANON MEDICAL SYSTEMS CORPORATION, Otawara-shi (JP)

(72) Inventors: Satoshi Matsunaga, Nasushiobara (JP); Yukifumi Kobayashi, Mitaka (JP)

(73) Assignee: CANON MEDICAL SYSTEMS CORPORATION, Otawara-shi (JP)

(21) Appl. No.: 16/406,487

(22) Filed: May 8, 2019

(30) Foreign Application Priority Data
May 9, 2018 (JP) 2018-090769

(51) Int. Cl. A61B 8/08; A61B 8/00

(52) U.S. Cl. CPC A61B 8/5207; A61B 8/466; A61B 5/065; A61B 8/483; A61B 8/5261

(57) ABSTRACT

An ultrasound diagnosis apparatus includes a processing circuitry. The processing circuitry is configured to perform a two-dimensional ultrasound scan on a subject via an ultrasound probe. The processing circuitry is configured to generate two-dimensional ultrasound image data on the basis of echo data acquired by the two-dimensional ultrasound scan. The processing circuitry is configured to reconstruct two-dimensional medical image data from three-dimensional medical image data of the subject, on the basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space. The processing circuitry is configured to calculate a degree of similarity between the two-dimensional ultrasound image data and the two-dimensional medical image data every time a condition is satisfied.

### CROSS-REFERENCE TO RELATED APPLICATIONS

This application is based upon and claims the benefit of priority from Japanese Patent Application No. 2018-090869, filed on May 9, 2018; the entire contents of which are incorporated herein by reference.

### FIELD

Embodiments described herein relate generally to an ultrasound diagnosis apparatus and a medical information processing method.

### BACKGROUND

Ultrasound diagnosis apparatuses are medical image diagnosis apparatuses configured to render a picture of the inside of an examined subject (hereinafter, “subject”) by transmitting and receiving an ultrasound wave to and from the subject. For example, an ultrasound diagnosis apparatus is configured to transmit ultrasound waves from an ultrasound probe brought into contact with the subject. The transmitted ultrasound waves are reflected by a tissue in the body of the subject and are received by the ultrasound probe as reflected-wave signals. Further, on the basis of the reflected-wave signals, an ultrasound image rendering a picture of the inside of the subject is generated.

In recent years, among such ultrasound diagnosis apparatuses, an ultrasound diagnosis apparatus is known to be configured to display, as a reference image, a Computed Tomography (CT) image, a Magnetic Resonance Imaging (MRI) image, or another ultrasound image that is on the same cross-sectional plane as that scanned by the ultrasound probe. The ultrasound diagnosis apparatus is configured to perform a registration process between the ultrasound image and the reference image by using position information of a position sensor attached to the ultrasound probe, so as to display the reference image that is on the same cross-sectional plane as that scanned by the ultrasound probe.

### BRIEF DESCRIPTION OF THE DRAWINGS

FIG. 1 is a block diagram illustrating an exemplary configuration of an ultrasound diagnosis apparatus according to a first embodiment;

FIG. 2 is a drawing for explaining an example of a process performed by a registration function according to the first embodiment;

FIG. 3A is a drawing illustrating an example of display control exercised by a controlling function according to the first embodiment;

FIG. 3B is a drawing illustrating another example of the display control exercised by the controlling function according to the first embodiment;

FIG. 4 is a drawing for explaining an example of an ultrasound volume data generating process performed under control of the controlling function according to the first embodiment; and

FIG. 5 is a flowchart illustrating a processing procedure performed by the ultrasound diagnosis apparatus according to the first embodiment.

### DETAILED DESCRIPTION

An ultrasound diagnosis apparatus includes a processing circuitry. The processing circuitry is configured to perform a two-dimensional ultrasound scan on a subject via an ultrasound probe. The processing circuitry is configured to generate two-dimensional ultrasound image data on the basis of echo data acquired by the two-dimensional ultrasound scan. The processing circuitry is configured to reconstruct two-dimensional medical image data from three-dimensional medical image data of the subject, on the basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space. The processing circuitry is configured to calculate a degree of similarity between the two-dimensional ultrasound image data and the two-dimensional medical image data every time a condition is satisfied.

In the following sections, exemplary embodiments of an ultrasound diagnosis apparatus and a medical information processing computer program (hereinafter, “medical information processing program”) will be explained. The exemplary embodiments described below are merely examples, and possible embodiments of the ultrasound diagnosis apparatus and the medical information processing program of the present disclosure are not limited to the explanations presented below.

### First Embodiment

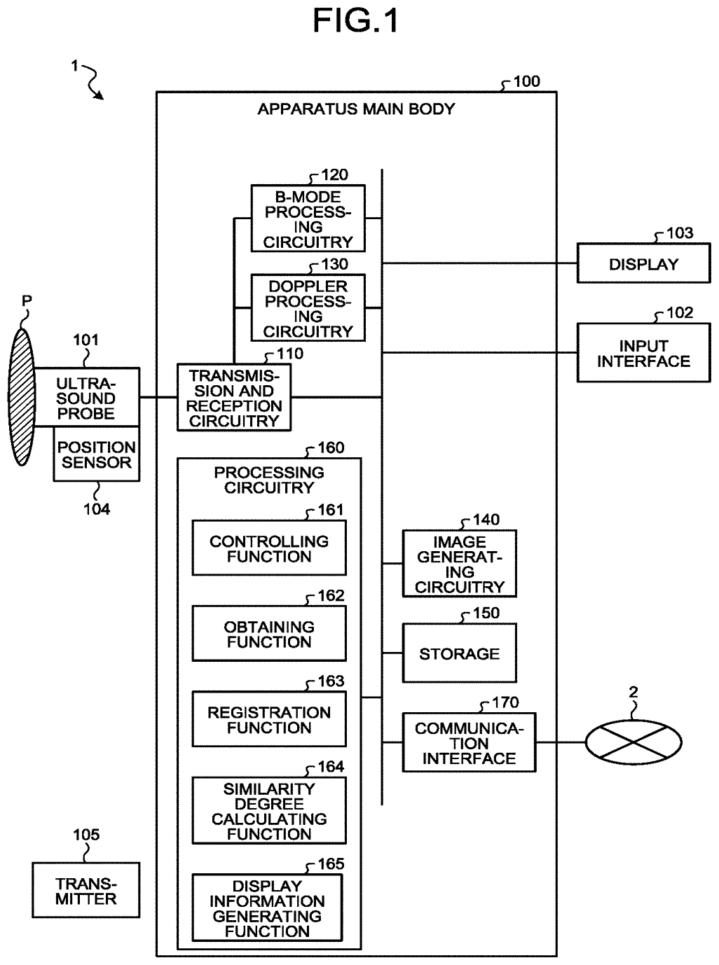

FIG. 1 is a block diagram illustrating an exemplary configuration of an ultrasound diagnosis apparatus 1 according to a first embodiment. As illustrated in FIG. 1, the ultrasound diagnosis apparatus 1 according to the first embodiment includes an apparatus main body 100, an ultrasound probe 101, an input interface 102, a display 103, a position sensor 104, and a transmitter 105. The ultrasound probe 101, the input interface 102, the display 103, the position sensor 104, and the transmitter 105 are connected to the apparatus main body 100 so as to be able to communicate therewith.

The ultrasound probe 101 includes a plurality of piezoelectric transducer elements. Each of the piezoelectric transducer elements is configured to generate an ultrasound wave on the basis of a drive signal supplied thereto from transmission and reception circuitry 110 included in the apparatus main body 100. Further, the ultrasound probe 101 is configured to receive reflected waves from a subject P and to convert the received reflected waves into electrical signals. In other words, the ultrasound probe 101 is configured to perform an ultrasound scan on the subject P and to receive the reflected waves from the subject P. Further, the ultrasound probe 101 includes a matching layer provided for the piezoelectric transducer elements, as well as a backing member or the like that prevents the ultrasound waves from propagating rearward from the piezoelectric transducer elements. The ultrasound probe 101 is detachably connected to the apparatus main body 100.

When an ultrasound wave is transmitted from the ultrasound probe 101 to the subject P, the transmitted ultrasound wave is repeatedly reflected on a surface of discontinuity of acoustic impedances at a tissue in the body of the subject P and is received as a reflected-wave signal by each of the plurality of piezoelectric transducer elements included in the ultrasound probe 101. The amplitude of the received reflected-wave signal is dependent on the difference between the acoustic impedances on the surface of discontinuity on which the ultrasound wave is reflected. When a transmitted ultrasound pulse is reflected on the surface of a moving blood flow, a cardiac wall, or the like, the reflected-wave signal is, due to the Doppler effect, subject to a frequency shift, depending on a velocity component of the moving members with respect to the ultrasound wave transmission direction.

In the present embodiment, the ultrasound probe 101 is a mechanical four-dimensional (4D) probe or a two-dimensional (2D) array probe capable of two-dimensionally scanning the subject P and also three-dimensionally scanning the subject P by using the ultrasound waves. The mechanical 4D probe is capable of performing the two-dimensional scan by using the plurality of piezoelectric transducer elements arranged in a row and is also capable of performing the three-dimensional scan by causing the plurality of piezoelectric transducer elements arranged in a row to swing with a predetermined angle (a swinging angle). Further, the 2D array probe is capable of performing the three-dimensional scan by using the plurality of piezoelectric transducer elements arranged in a matrix formation and is also capable of performing the two-dimensional scan by transmitting and receiving ultrasound waves in a converged manner. In addition, the 2D array probe is also capable of performing a two-dimensional scan on a plurality of cross-sectional planes at the same time.

The input interface 102 includes a mouse, a keyboard, a button, a panel switch, a touch command screen, a wheel, a dial, a foot switch, a trackball, a joystick, and/or the like. The input interface 102 is configured to receive various types of setting requests from an operator of the ultrasound diagnosis apparatus 1 and to transfer the received various types of setting requests to the apparatus main body 100.

The display 103 is configured to display a Graphical User Interface (GUI) used by the operator of the ultrasound diagnosis apparatus 1 for inputting the various types of setting requests via the input interface 102 and to display ultrasound image data generated by the apparatus main body 100 and the like. Further, the display 103 is configured to display various types of messages and display information to inform the operator of processing statuses and processing results of the apparatus main body 100. Further, the display 103 includes a speaker and is capable of outputting audio.

The position sensor 104 and the transmitter 105 are devices (a position detecting system) configured to obtain position information of the ultrasound probe 101. For example, the position sensor 104 may be a magnetic sensor attached to the ultrasound probe 101. Further, for example, the transmitter 105 is a device arranged in an arbitrary position and configured to form a magnetic field centered on the device and spreading outwardly.

The position sensor 104 is configured to detect the three-dimensional magnetic field formed by the transmitter 105. Further, the position sensor 104 is configured to calculate the position (coordinates) and the orientation (an angle) thereof in a space defined by using the transmitter 105 as the origin, on the basis of information about the detected magnetic field and to further transmit the calculated position and orientation to processing circuitry 160 (explained later). The three-dimensional position information (the position and the orientation) of the position sensor 104 transmitted to the processing circuitry 160 is used after being converted, as appropriate, into either position information of the ultrasound probe 101 or position information of a scanned range scanned by the ultrasound probe 101.

For example, the position information of the position sensor 104 may be converted into the position information of the ultrasound probe 101, on the basis of a positional relationship between the position sensor 104 and the ultrasound probe 101. Further, the position information of the ultrasound probe 101 may be converted into the position information of the scanned range on the basis of a positional relationship between the ultrasound probe 101 and the scanned range. In addition, the position information of the scanned range may also be converted into pixel positions, on the basis of a positional relationship between the scanned range and sampling points on scanned lines. In other words, it is possible to convert the three-dimensional position information of the position sensor 104 into the pixel positions of the ultrasound image data taken by the ultrasound probe 101.

The present embodiment is also applicable to situations where the position information of the ultrasound probe 101 is obtained by using a system other than the position detecting system described above. For example, the present embodiment may be configured so as to obtain the position information of the ultrasound probe 101 by using a gyro sensor, an acceleration sensor, or the like.

The apparatus main body 100 is an apparatus configured to generate the ultrasound image data on the basis of the reflected-wave signals received by the ultrasound probe 101. The apparatus main body 100 illustrated in FIG. 1 is an apparatus capable of generating two-dimensional ultrasound image data on the basis of two-dimensional reflected-wave data (echo data) received by the ultrasound probe 101. Further, the apparatus main body 100 illustrated in FIG. 1 is an apparatus capable of generating three-dimensional ultrasound image data (ultrasound volume data) on the basis of three-dimensional reflected-wave data received by the ultrasound probe 101.

As illustrated in FIG. 1, the apparatus main body 100 includes the transmission and reception circuitry 110, B-mode processing circuitry 120, Doppler processing circuitry 130, image generating circuitry 140, a storage 150, the processing circuitry 160, and a communication interface 170. The transmission and reception circuitry 110, the B-mode processing circuitry 120, the Doppler processing circuitry 130, the image generating circuitry 140, the storage 150, the processing circuitry 160, and the communication interface 170 are connected together so as to be able to communicate with one another. Further, the apparatus main body 100 is connected to a network 2.

The transmission and reception circuitry 110 includes a pulse generator, a transmission delay unit, a puller, and the like and is configured to supply the drive signal to the ultrasound probe 101. The pulse generator is configured to repeatedly generate a rate pulse used for forming a transmission ultrasound wave at a predetermined rate frequency. Further, the transmission delay unit is configured to apply a delay period that is required to converge the ultrasound waves generated by the ultrasound probe 101 into the form of a beam and to determine transmission directionality and that corresponds to each of the piezoelectric transducer elements, to each of the rate pulses generated by the pulse generator. Further, the pulser is configured to apply the drive signal (a drive pulse) to the ultrasound probe 101 with timing based on the rate pulses. In other words, by varying the delay periods applied to the rate pulses, the transmission delay unit is able to arbitrarily adjust the transmission directions of the ultrasound waves transmitted from the surfaces of the piezoelectric transducer elements.

In this situation, the transmission and reception circuitry 110 has a function that is able to instantly change the transmission frequency, the transmission drive voltage, and the like, for the purpose of executing a predetermined scan sequence on the basis of an instruction from the processing circuitry 160 (explained later). In particular, the function to change the transmission drive voltage is realized by using a linear-amplifier-type transmission circuit of which the value can be instantly switched or by using a mechanism configured to electrically switch between a plurality of power source units.

Further, the transmission and reception circuitry 110 includes a pre-amplifier, an Analog/Digital (A/D) converter, a reception delay unit, an adder, and the like and is configured to generate reflected-wave data by performing various types of processes on the reflected-wave signals received by the ultrasound probe 101. The pre-amplifier is configured to amplify the reflected-wave signals for each of the channels. The A/D converter is configured to perform an A/D conversion process on the amplified reflected-wave signals. The reception delay unit is configured to apply a delay period required to determine reception directionality, to the result of the A/D conversion. The adder is configured to generate the reflected-wave data by performing an adding process on the reflected-wave signals processed by the reception delay unit. As a result of the adding process performed by the adder, reflected components from the direction corresponding to the reception directionality of the reflected-wave signals are emphasized, so that a comprehensive beam used in the ultrasound transmission and reception is formed on the basis of the reception directionality and the transmission directionality.

When the subject P is to be two-dimensionally scanned, the transmission and reception circuitry 110 is configured to cause a two-dimensional ultrasound beam to be transmitted from the ultrasound probe 101. Further, the transmission and reception circuitry 110 is configured to generate two-dimensional reflected-wave data from two-dimensional reflected-wave signals received by the ultrasound probe 101. In contrast, when the subject P is to be three-dimensionally scanned, the transmission and reception circuitry 110 of the present embodiment is configured to cause a three-dimensional ultrasound beam to be transmitted from the ultrasound probe 101. Further, the transmission and reception circuitry 110 is configured to generate three-dimensional reflected-wave data from three-dimensional reflected-wave signals received by the ultrasound probe 101.

In this situation, the output signal from the transmission and reception circuitry 110 may be in a form selected from among various forms such as being a signal called a Radio Frequency (RF) signal including phase information or being amplitude information obtained after an envelope detecting process.

The B-mode processing circuitry 120 is configured to receive the reflected-wave data from the transmission and reception circuitry 110 and to generate data (B-mode data) in which signal intensities are expressed with levels of brightness, by performing a logarithmic amplification process, an envelope detecting process, and/or the like thereon.

The Doppler processing circuitry 130 is configured to generate data (Doppler data) obtained by extracting moving member information such as velocity, dispersion, power, and the like with respect to multiple points, by performing a frequency analysis to obtain velocity information from the reflected-wave data received from the transmission and reception circuitry 110 and extracting a blood flow, a tissue, and a contrast agent echo component influenced by the Doppler effect.

The B-mode processing circuitry 12.0 and the Doppler processing circuitry 130 illustrated in FIG. 1 are capable of processing both the two-dimensional reflected-wave data and the three-dimensional reflected-wave data. In other words, the B-mode processing circuitry 120 is configured to generate two-dimensional B-mode data from the two-dimensional reflected-wave data and generate three-dimensional B-mode data from the three-dimensional reflected-wave data. Further, the Doppler processing circuitry 130 is configured to generate two-dimensional Doppler data from the two-dimensional reflected-wave data and to generate three-dimensional Doppler data from the three-dimensional reflected-wave data.

The image generating circuitry 140 is configured to generate ultrasound image data from the data generated by the B-mode processing circuitry 120 and the Doppler processing circuitry 130. In other words, the image generating circuitry 140 is configured to generate two-dimensional B-mode image data in which intensities of the reflected waves are expressed with levels of brightness, from the two-dimensional B-mode data generated by the B-mode processing circuitry 120. Further, the image generating circuitry 140 is configured to generate two-dimensional Doppler image data expressing moving member information, from the two-dimensional Doppler data generated by the Doppler processing circuitry 130. The two-dimensional Doppler image data may be a velocity image, a dispersion image, a power image, or an image combining any of these types of images. Further, the image generating circuitry 140 is also capable of generating M-mode image data from time-series data of B-mode data on a scanning line generated by the B-mode processing circuitry 120. Further, the image generating circuitry 140 is also capable of generating a Doppler waveform obtained by plotting pieces of velocity information of a blood flow or a tissue along a time series, from the Doppler data generated by the Doppler processing circuitry 130.

In this situation, generally speaking, the image generating circuitry 140 converts (by performing a scan convert process) a scanning line signal sequence from an ultrasound scan into a scanning line signal sequence in a video format used by, for example, television and generates display-purpose ultrasound image data. More specifically, the image generating circuitry 140 generates the display-purpose ultrasound image data by performing a coordinate transformation process compliant with the ultrasound scan mode used by the ultrasound probe 101. Further, as various types of image processing processes besides the scan convert process, the image generating circuitry 140 performs, for example, an image processing process (a smoothing process) to re-generate a brightness average value image, an image processing process (an edge enhancement process) that uses a differential filter inside an image, or the like, by using a plurality of image frames resulting from the scan convert process. Also, the image generating circuitry 140 combines text information of various types of parameters, scale graduations, body marks, and the like with the ultrasound image data.

In other words, the B-mode data and the Doppler data are each ultrasound image data before the scan convert process. The data generated by the image generating circuitry 140 is the display-purpose ultrasound image data after the scan convert process. The B-mode data and the Doppler data may also be referred to as raw data. The image generating circuitry 140 is configured to generate “two-dimensional B-mode image data and two-dimensional Doppler image data” serving as display-purpose two-dimensional ultrasound image data, from “two-dimensional B-mode data and two-dimensional Doppler data” represented by the two-dimensional ultrasound image data before the scan convert process.

Further, the image generating circuitry 140 is configured to generate three-dimensional B-mode image data by performing a coordinate transformation process on the three-dimensional B-mode data generated by the B-mode processing circuitry 120. Further, the image generating circuitry 140 is configured to generate three-dimensional Doppler image data by performing a coordinate transformation process on the three-dimensional Doppler data generated by the Doppler processing circuitry 130. In other words, the image generating circuitry 140 is configured to generate the “three-dimensional B-mode image data and three-dimensional Doppler image data” as “three-dimensional ultrasound image data (volume data)”.

Further, to generate various types of two-dimensional image data used for displaying ultrasound volume data on the display 103, the image generating circuitry 140 is configured to perform a rendering process on the ultrasound volume data. An example of the rendering process performed by the image generating circuitry 140 is a process of reconstructing Multi Planar Reconstruction (MPR) image data from the ultrasound volume data by implementing an MPR method. Further, other examples of the rendering process performed by the image generating circuitry 140 are a process of applying a “Curved MPR” on the ultrasound volume data and a process of applying “Maximum Intensity Projection” on the ultrasound volume data. Also, other examples of the rendering process performed by the image generating circuitry 140 are a Volume Rendering (VR) process and a Surface Rendering (SF) process to generate two-dimensional image data reflecting three-dimensional information. The image generating circuitry 140 is an example of the processing circuitry.

The storage 150 is a memory configured to store therein the display-purpose ultrasound image data generated by the image generating circuitry 140. Further, the storage 150 is also capable of storing therein any of the data generated by the B-mode processing circuitry 120 and the Doppler processing circuitry 130. The operator is able to invoke any of the B-mode data and the Doppler data stored in the storage 150 after a diagnosing process, for example. The invoked B-mode data and Doppler data can serve as the display-purpose ultrasound image data after being routed through the image generating circuitry 140.

Further, the storage 150 is configured to store therein control programs for performing ultrasound transmissions and receptions, image processing processes, and display processes as well as various types of data such as diagnosis information (e.g., subjects' IDs, medical doctors' observations), diagnosis protocols, various types of body marks, and the like. Further, the data stored in the storage 150 may be transferred to an external apparatus via an interface (not illustrated). The external apparatus may be, for example, a Personal Computer (PC) used by the operator (e.g., a medical doctor) who performs an image diagnosis process, a storage medium such as a Compact Disk (CD) or Digital Versatile Disk (DVD), a printer, or the like.

The processing circuitry 160 is configured to control overall processes performed by the ultrasound diagnosis apparatus 1. More specifically, on the basis of the various types of setting requests input from the operator via the input interface 102 and various types of control programs and various types of data read from the storage 150, the processing circuitry 160 is configured to control processes performed by the transmission and reception circuitry 110, the B-mode processing circuitry 120, the Doppler processing circuitry 130, and the image generating circuitry 140. Further, the processing circuitry 160 is configured to exercise control so that any of the display-purpose ultrasound image data stored in the storage 150 is displayed on the display 103. In the following sections, the ultrasound image data displayed on the display 103 may also be referred to as ultrasound images.

The communication interface 170 is an interface used for communicating with any of various types of apparatuses provided in the hospital via the network 2. Through the communication interface 170, the processing circuitry 160 is configured to communicate with external apparatuses. For example, the processing circuitry 160 receives medical image data (e.g., Computed Tomography [CT] image data, Magnetic Resonance Imaging [MRI] image data) taken by any medical image diagnosis apparatus other than the ultrasound diagnosis apparatus 1, via the network 2. Further, the processing circuitry 160 causes the display 103 to display the received medical image data together with the ultrasound image data taken by the ultrasound diagnosis apparatus 1. The displayed medical image data may be one or more images on which the image generating circuitry 140 has performed an image processing process (the rendering process). Further, the medical image data displayed together with the ultrasound image data may be obtained via a storage medium such as a Compact Disk Read-Only Memory (CD-ROM), a Magneto-Optical (MO) disk, a DVD, or the like.

Further, the processing circuitry 160 executes a controlling function 161, an obtaining function 162, a registration function 163, a similarity degree calculating function 164, and a display information generating function 165. The processing circuitry 160 is an example of the processing circuitry. Details of the functions executed by the processing circuitry 160 will be explained later.

In this situation, for example, the processing functions executed by the constituent elements of the processing circuitry 160 illustrated in FIG. 1, namely, the controlling function 161, the obtaining function 162, the registration function 163, the similarity degree calculating function 164, and the display information generating function 165 are recorded in the storage 150 in the form of computer-executable programs. The processing circuitry 160 is a processor configured to realize the functions corresponding to the programs by reading and executing the programs from the storage 150. In other words, the processing circuitry 160 that has read the programs has the functions illustrated within the processing circuitry 160 in FIG. 1.

In the present embodiment, the example is explained in which the single processing circuit (the processing circuitry 160) realizes the processing functions described below; however, another arrangement is also acceptable in which the processing circuitry is structured by combining together a plurality of independent processors, so that the functions are realized as a result of the processors executing the programs.

The term “processor” used in the above explanation denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processors realize the functions by reading and executing the programs saved in the storage 150. In this situation, instead of saving the programs in the storage 150, it is also acceptable to directly incorporate the programs in the circuits of the processors. In that situation, the processors realize the functions thereof by reading and executing the programs incorporated in the circuits thereof. The processors in the present embodiment do not each necessarily have to be structured as a single circuit. It is also acceptable to structure one processor by combining together a plurality of independent circuits so as to realize the functions thereof. Further, it is also acceptable to integrate two or more of the constituent elements in any of the drawings into one processor so as to realize the functions thereof.

An overall configuration of the ultrasound diagnosis apparatus 1 according to the first embodiment has thus been explained. The ultrasound diagnosis apparatus 1 according to the present embodiment structured as described above is configured to make it possible to improve efficiency of medical examinations by maintaining a certain level of precision for the registration processes. As explained above, ultrasound diagnosis apparatuses are configured to perform the registration process between ultrasound images and reference images (e.g., CT images, MRI images) by using the position information of the position sensor attached to the ultrasound probe and are capable of displaying the reference images that is on the same cross-sectional plane as that scanned by the ultrasound probe.

In this situation, between an ultrasound image and a reference image, it would be difficult to exactly match each other even by performing the registration process, because of a difference in the postures of the subject at the times of acquisition and because of errors of the position sensor. In other words, even when the registration process is performed between the ultrasound image and the reference image, there may be a misalignment between the images in some situations. In those situations, when an ultrasound scan is performed while the ultrasound probe is moved along the body surface, the amount of misalignment between the images might increase, and the efficiency of the medical examination might be degraded due to a decrease in the level of precision of the registration process. To cope with this situation, the ultrasound diagnosis apparatus 1 according to the first embodiment is configured to monitor the level of precision of the registration processes and to perform a re-registration process when the level of precision of the registration processes becomes lower and is thus able to improve the efficiency of the medical examination by maintaining a certain level of precision for the registration processes. In the following sections, details of the processes performed by the ultrasound diagnosis apparatus 1 will be explained.

The controlling function 161 is configured to control the entirety of the ultrasound diagnosis apparatus 1. For example, by controlling the transmission and reception circuitry 110, the B-mode processing circuitry 120, and the Doppler processing circuitry 130, the controlling function 161 is configured to control the acquisition of the reflected-wave data and the generation of the B-mode data and the Doppler data. In other words, the controlling function 161 causes a two-dimensional ultrasound scan and a three-dimensional ultrasound scan to be performed on the subject, via the ultrasound probe 101 provided with the position sensor 104.

Further, the controlling function 161 is configured to generate ultrasound image data by controlling processes performed by the image generating circuitry 140. Further, the controlling function 161 is configured to obtain, via the network 2, medical image data (e.g., CT image data, MRI image data) taken by a medical image diagnosis apparatus other than the ultrasound diagnosis apparatus 1. For example, the controlling function 161 obtains medical image data designated via the input interface 102 (e.g., volume data acquired by a medical image diagnosis apparatus other than the ultrasound diagnosis apparatus 1) from the medical image diagnosis apparatus or an image storing apparatus in the network 2. In one example, the controlling function 161 obtains CT volume data acquired through an image taking process performed by an X-ray CT apparatus on the subject from whom ultrasound image data is acquired while reference images are being referenced. In the following sections, such volume data from which reference images are generated may also be referred to as reference volume data.

Further, the controlling function 161 is configured to exercise control so that the display 103 displays the obtained medical image data and ultrasound image data. For example, the controlling function 161 causes the display 103 to display an MPR image reconstructed from the reference volume data and a display-purpose ultrasound image generated by the image generating circuitry 140. In this situation, the reconstruction of the MPR image from the reference volume data is performed by the image generating circuitry 140.

The obtaining function 162 is configured to obtain probe position information indicating the position and the orientation of the ultrasound probe 101. For example, the obtaining function 162 obtains pieces of probe position information over a plurality of temporal phases. In one example, the obtaining function 162 chronologically receives pieces of position information of the position sensor 104 from the position sensor 104. The pieces of position information of the position sensor 104 are used as being converted, as appropriate, into pieces of probe position information. For example, the pieces of position information of the position sensor 104 are converted into the pieces of probe position information on the basis of a positional relationship between the position sensor 104 and the ultrasound probe 101. Each of the pieces of probe position information is information indicating the coordinates of the ultrasound probe 101 in real space and the position and the angle (a posture) of the ultrasound probe 101 at the coordinates.

For example, when a magnetic sensor is used as the position sensor 104, an initial position of the ultrasound probe 101 is set in the three-dimensional magnetic field formed by the transmitter 105. For example, the operator holds the ultrasound probe 101 to which the position sensor 104 is attached so as to be positioned perpendicular to the body surface of the subject P and presses an initial position setting button while the ultrasound probe 101 is kept in that state. When having received the pressing of the initial position setting button, the obtaining function 162 sets the probe position information at that time as an initial position. Further, the obtaining function 162 obtains displacement amounts in the position and the orientation of the ultrasound probe 101 in each of the temporal phases (at each of different times), on the basis f differences between the pieces of probe position information in the plurality of temporal phases that are chronologically obtained and the initial position.

In this manner, the obtaining function 162 obtains he pieces of probe position information in a time series. Further, the obtaining function 162 stores the pieces of probe position information in the time series into the storage 150 so as to be kept in correspondence with obtainment times of the pieces of probe position information. The obtainment times are used for keeping the pieces of probe position information in correspondence with pieces of ultrasound image data. In other words, the processing circuitry 160 is able to specify the position and the orientation of the ultrasound probe 101 at the time when a desired ultrasound image was taken, by referencing a piece of probe position information obtained at the time coinciding with the time at which the ultrasound image data was taken.

The registration function 163 is configured to perform the registration process between the ultrasound image data and the reference volume data. More specifically, the registration function 163 determines a correspondence relationship between a three-dimensional space (a first coordinate space) in which the ultrasound image data was acquired and another three-dimensional space (a second coordinate space) in which the reference volume data was acquired. In other words, the registration function 163 determines a position (coordinates) in the second coordinate space corresponding to a position (coordinates) of the ultrasound image data in the first coordinate space. In this situation, the registration function 163 determines a correspondence relationship between the position information of the ultrasound probe 101 and the reference volume data, by determining a correspondence relationship between the ultrasound image data and the reference volume data.

In one example, the registration function 163 arranges a site included in the ultrasound image data to be substantially in the same position with the corresponding site in the reference volume data and further determines the position of the ultrasound image data in the coordinate space (the second coordinate space) of the reference volume data at that time. In this situation, the ultrasound image data is kept in correspondence with the position information of the ultrasound probe 101. Thus, by using the correspondence information, the registration function 163 determines the correspondence relationship between the position information of the ultrasound probe 101 and the reference volume data.

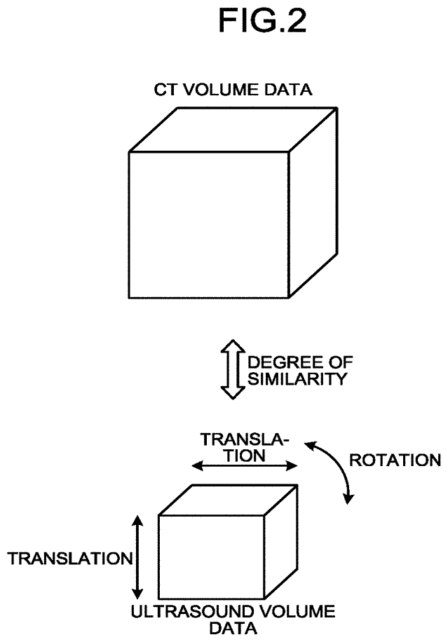

FIG. 2 is a drawing for explaining an example of a process performed by the registration function 163 according to the first embodiment. With reference to FIG. 2, an example will be explained in which CT volume data is used as the reference volume data. Further, with reference to FIG. 2, the example will be explained in which a registration process is performed by using ultrasound volume data as the ultrasound image data. For example, as illustrated in FIG. 2, the registration function 163 determines a correspondence relationship between the CT volume data and the ultrasound volume data, by calculating a degree of similarity between the CT volume data and the ultrasound volume data and searching for the correspondence relationship until the calculated degree of similarity reaches a predetermined value.

In one example, the registration function 163 first arbitrarily brings the coordinates of the CT volume data into correspondence with the coordinates of the ultrasound volume data. Further, as illustrated in FIG. 2, the registration function 163 diversely varies the position of the ultrasound volume data with respect to the CT volume data by translating and rotating the ultrasound volume data and calculates a degree of similarity between the two pieces of data in each of the positions. Further, the registration function 163 determines the position of the ultrasound volume data with respect to the CT volume data at the time when the calculated degree of similarity reaches the predetermined value as the correspondence relationship between the two pieces of data. In other words, the registration function 163 applies a transformation matrix to one of the pieces of data so that the degree of similarity between the two pieces of data exceeds the predetermined value (for example, becomes maximized) and further extracts the transformation matrix observed when the degree of similarity reaches the predetermined value. Further, the registration function 163 brings the coordinates of the ultrasound volume data to which the extracted transformation matrix was applied and the coordinates of the CT volume data into correspondence with the initial correspondence relationship that was arbitrarily brought into correspondence initially and determines the correspondence as the correspondence relationship. The registration function 163 stores information about the extracted transformation matrix and the correspondence relationship into the storage 150.

In this situation, because the ultrasound volume data is kept in correspondence with the position information of the ultrasound probe 101 observed at the time of the acquisition, the registration function 163 is able to determine the correspondence relationship between the coordinate space of the CT volume data and the position information of the ultrasound probe 101. For example, the ultrasound volume data is reconstructed from a plurality of pieces of two-dimensional ultrasound image data kept in correspondence with the position information of the ultrasound probe 101. Accordingly, the position information of the ultrasound probe 101 is kept in correspondence with the positions to which the pieces of two-dimensional ultrasound image data correspond in the coordinate space of the CT volume data.

In this situation, to calculate the degree of similarity, the registration function 163 may perform the calculation by using mutual information between the two pieces of data, for example. In that situation, the registration function 163 applies various types of transformation matrices to the ultrasound volume data and calculates a mutual information value for each of the transformation matrices. Further, the registration function 163 determines such a transformation matrix that makes the mutual information exceed a predetermined value as the correspondence relationship between the pieces of data. Alternatively, as an index indicating the degree of similarity, the registration function 163 may use any arbitrary index other than the mutual information.

Further, the registration function 163 is capable of performing not only the registration processes described above but also other various types of registration processes. For example, the registration function 163 may extract the shape of a predetermined site (e.g., an organ or a blood vessel) from the pieces of volume data and perform a registration process by using a degree of similarity between the extracted sites.

As explained above, as a result of the registration function 163 performing the registration process, the controlling function 161 is able to display the reference image that is substantially in the same position as the position scanned by the ultrasound probe 101. In other words, the controlling function 161 specifies the positions of the acquired pieces of two-dimensional ultrasound image data in the coordinate space of the ultrasound volume data used in the registration process, on the basis of the positions of the ultrasound probe 101 corresponding to the acquired pieces of two-dimensional ultrasound image data. Further, the controlling function 161 applies the abovementioned transformation matrix to the coordinates of the specified positions and further extracts the coordinates of the CT volume data corresponding to the coordinates resulting from the transformation matrix on the basis of the information about the correspondence relationship. Subsequently, the controlling function 161 controls the image generating circuitry 140 so as to generate the tomography images (the CT images) at the extracted coordinates of the CT volume data and further controls the display 103 so as to display the generated CT images.

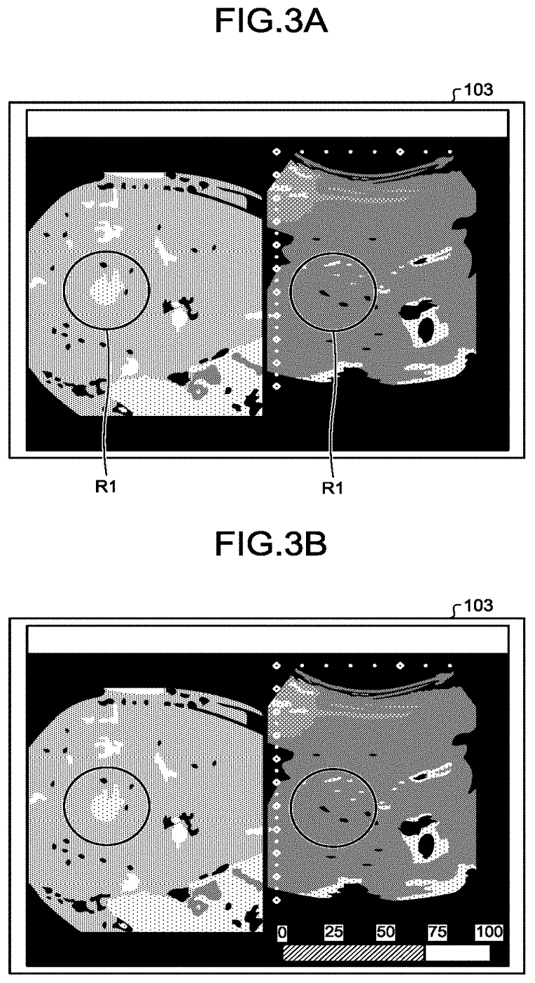

FIG. 3A is a drawing illustrating an example of display control exercised by the controlling function 161 according to the first embodiment. In FIG. 3A, a CT image is displayed on the left side of the display 103, whereas an ultrasound image is displayed on the right side. As indicated on the right side of FIG. 3A, the controlling function 161 causes the display 103 to display the ultrasound image corresponding to the position of the ultrasound scan performed by the ultrasound probe 101. Further, as explained above, the controlling function 161 generates a CT image corresponding to the position in which the ultrasound scan was performed by the ultrasound probe 101 and fort causes the display 103 to display the generated CT image as indicated on the left side of FIG. 3A. In this situation, because the registration process is performed by the registration function 163, the ultrasound image and the CT image displayed on the display 103 are images that are substantially in the same position as each other and include mutually the same region of interest R1, as illustrated in FIG. 3A. In this situation, in accordance with various positions in which the ultrasound scan is performed by the ultrasound probe 101, ultrasound images and CT images will be displayed as being updated.

As illustrated in FIG. 3A, when the manipulation is performed while having displayed the reference image that is substantially in the same positions as the ultrasound image, the amount of misalignment between the images may increase in some situations, and the efficiency of the medical examination may be degraded due to a decrease in the level of precision of the registration process. To cope with this situation, the ultrasound diagnosis apparatus 1 according to the present embodiment is configured to periodically observe the degree of similarity between the images and to exercise control so as to perform a registration process again when the degree of similarity becomes smaller. Accordingly, the ultrasound diagnosis apparatus 1 thus maintains a certain level of precision for the registration processes and improves the efficiency of the medical examination.

More specifically, every time a predetermined condition is satisfied, the similarity degree calculating function 164 is configured to calculate a degree of similarity between the two-dimensional ultrasound image data and the two-dimensional medical image data. Even more specifically, every time the predetermined condition is satisfied, the similarity degree calculating function 164 calculates the degree of similarity between the ultrasound image data acquired by the ultrasound scan performed by the ultrasound probe 101 and the reference image that is in the position specified by using the transformation matrix. In other words, the similarity degree calculating function 164 calculates the degree of similarity used for judging the degree of positional misalignments between the sequentially-generated ultrasound images and the reference images that are substantially in the same position as the ultrasound images and are generated on the basis of the correspondence relationship determined by the registration process.

In this situation, for example, the similarity degree calculating function 164 calculates the abovementioned degree of similarity once every predetermined periodic cycle. In one example, the similarity degree calculating function 164 calculates the degree of similarity once every predetermined time interval (e.g., once every 50 ms or once every 100 ms) or once every predetermined frame interval (e.g., once every 10 frames). In another example, the similarity degree calculating function 164 calculates the degree of similarity every time the ultrasound probe 101 has moved a predetermined distance. In this situation, the periodic cycle used for calculating the degrees of similarity may be varied in accordance with at least one selected from among: the target site, the physique of the subject, and the posture of the subject during the scan. In other words, the periodic cycle used for calculating the degrees of similarity is set in accordance with at least one selected from among the site, the physique, and the posture. For example, the similarity degree calculating function 164 may shorten or extend the periodic cycle when the target site is an organ that involves movements such as heart or a lung or an organ that changes the position and the shape thereof due to the movements. In other words, with respect to regions that involve movements due to heartbeats or respiration, the similarity degree calculating function 164 is able to change, as appropriate, the periodic cycle used for calculating the degrees of similarity on the basis of the manner in which each region moves and the periodic cycle in which each region moves. For instance, examples the organ that changes he position and the shape thereof due to movements of another organ such as the heart or a lung that involves movements include the liver. In one example, it is known that when the subject is in a supine posture, the liver moves upward in the vertical direction during inhalation and moves downward in the vertical direction during exhalation.

Further, for example, when the subject has a larger physique and the distance (the depth) from the body surface to the target site is longer, the similarity degree calculating function 164 may shorten the periodic cycle used for calculating the degrees of similarity. Conversely, when the subject has a smaller physique and the distance from the body surface to the target site is shorter, the similarity degree calculating function 164 may extend the periodic cycle.

Further, for example, even when the target site is the same, the similarity degree calculating function 164 may extend or shorten the periodic cycle used for calculating the degrees of similarity, depending on differences in the posture of the subject. In one example, when the subject is in a posture in which body movements do not easily occur, the similarity degree calculating function 164 may extend the periodic cycle used for calculating the degrees of similarity. Conversely, when the subject is in a posture in which body movements easily occur, the similarity degree calculating function 164 may shorten the periodic cycle.

Further, the similarity degree calculating function 164 is able to set an arbitrary periodic cycle in accordance with any of various combinations of the site, the physique, and the posture.

Further, for example, the similarity degree calculating function 164 may calculate the degree of similarity when the condition is satisfied where pressure is applied to the subject by the ultrasound probe 101. In one example, the similarity degree calculating function 164 calculates the degree of similarity when the condition is satisfied where pressure is applied to a tissue of the subject to perform an ultrasound elastography process by which a distribution of firmness levels in the tissue is expressed in an image by using ultrasound waves.

Further, for example, as the degree of similarity, the similarity degree calculating function 164 may calculate mutual information between the ultrasound image and the reference image. In other words, similarly to the registration function 163 described above, the similarity degree calculating function 164 may calculate a mutual information value as the degree of similarity between the images. In this situation, the images of which the degree of similarity is calculated are used in an arbitrary combination. For example, when a two-dimensional ultrasound image is displayed, while an MPR image reconstructed from reference volume data is displayed as a reference image corresponding thereto, the similarity degree calculating function 164 may calculate, once every predetermined periodic cycle, a degree of similarity between the two-dimensional ultrasound image and the MPR image reconstructed from the reference volume data.

In another example, when both an ultrasound image and a reference image are displayed as MPR images, the similarity degree calculating function 164 may calculate, once every predetermined periodic cycle, a degree of similarity between the MPR image reconstructed from the ultrasound volume data and the MPR image reconstructed froze reference volume data.

In yet another example, when ultrasound image data is three-dimensionally acquired, the similarity degree calculating function 164 may calculate, once every predetermined periodic cycle, a degree of similarity between ultrasound volume data and reference volume data.

In this situation, the similarity degree calculating function 164 may calculate the degree of similarity with respect to the entirety of the pieces of image data or may calculate the degree of similarity with respect to parts of the pieces of image data. For example, the similarity degree calculating function 164 may extract the shape of a tumor section, a marked section, a focused section, a central part of the image, or the like from each of the pieces of image data and calculate a degree of similarity between the images by using the extracted shapes. In other words, the similarity degree calculating function 164 may calculate the degree of similarity by narrowing the processing targets to the regions of interest in the images.

In this situation, for example, the tumor section may be designated by an operator (e.g., a medical doctor) from within the images or may automatically be extracted by the similarity degree calculating function 164. When tumor sections are automatically extracted, for example, the similarity degree calculating function 164 extracts a tumor section from each of the pieces of image data by performing a pattern matching process or the like and further calculates a degree of similarity between the images in the extracted tumor sections.

Further, for example, the marked section is a region designated by the user from each of the ultrasound and reference images. In that situation, the similarity degree calculating function 164 calculates a degree of similarity of the marked sections between the images. In another example, the similarity degree calculating function 164 may extract a focused section from the ultrasound image on the basis of information about a focus contained in an acquisition condition of the ultrasound image and further calculate a degree of similarity between the extracted focused section and a position within the reference image corresponding to the focused section. In yet another example, the similarity degree calculating function 164 may extract a central part from each of the ultrasound and reference images and further calculate a degree of similarity between the extracted central parts.

As explained above, the similarity degree calculating function 164 is configured to calculate the degree of similarity between the ultrasound image and the reference image. In this situation, the similarity degree calculating function 164 may also normalize the calculated degree of similarity. For example, the similarity degree calculating function 164 normalizes the calculated degree of similarity, by using, as a reference, a degree of similarity between the ultrasound image and the reference image observed when the correspondence relationship between the first coordinate space and the second coordinate space was determined. More specifically, for example, the similarity degree calculating function 164 calculates relative values of degrees of similarity between sequentially-generated ultrasound images and corresponding reference images, by expressing the degree of similarity observed when the correspondence relationship between the first coordinate space and the second coordinate space was initially determined, as “100”.

Returning to the description of FIG. 1, the display information generating function 165 is configured to generate display information displayed by the display 103 under the control of the controlling function 161. For example, the display information generating function 165 generates display information used for displaying the degrees of similarity calculated by the similarity degree calculating function 164. In one example, the display information generating function 165 generates an indicator indicating the degrees of similarity. In this situation, for example, the display information generating function 165 may generate an indicator indicating the degrees of similarity as absolute values or may generate an indicator indicating the degrees of similarity as normalized values.

When the degree of similarity has been calculated by the similarity degree calculating function 164 in the manner described above, the controlling function 161 causes the display 103 to display the calculated degree of similarity. For example, the controlling function 161 causes the display 103 to display the display information generated by the display information generating function 165. FIG. 32 is a drawing illustrating another example of the display control exercised by the controlling function 161 according to the first embodiment. As illustrated in FIG. 3B, the controlling function 161 may display an indicator indicating the degree of similarity on the screen displaying the ultrasound image and the reference image. In this situation, the indicator illustrated in FIG. 3B is an indicator indicating the degree of similarity as a normalized value. In other words, the indicator is displayed so as to display a relative value of the degree of similarity between each of the sequentially-generated ultrasound images and a corresponding reference image, while expressing the degree of similarity between the ultrasound image and the reference image observed immediately after the registration process as “100”.

In the manner described above, by displaying chronological changes in the degree of similarity together with the images, the ultrasound diagnosis apparatus 1 according to the first embodiment is able to help the user understand at all times the state of the position alignments between the ultrasound image and the reference image. As a result, the user is able to immediately notice when the level of precision of the registration process becomes lower (when the degree of similarity between the images becomes smaller) and is thus able to correct the position alignment. For example, the user is able to monitor the state of the position alignments by referencing the indicator displayed on the display 103 and, when the degree of similarity becomes smaller than a predetermined value, the user is able to instruct that a registration process be performed again by operating the input interface 102. With these arrangements, the registration function 163 is able to correct the position alignment between the ultrasound image and the reference image so as to have an appropriate correspondence relationship.

In this situation, for example, the controlling function 161 may cause the display 103 to display information indicating that the degree of similarity has become smaller than the predetermined value and a GUI used for having the registration process performed again. In that situation, for example, the display information generating function 165 generates alert information indicating that the degree of similarity is smaller than the predetermined value. After that, when the degree of similarity becomes smaller than the predetermined value, the controlling function 161 arranges the generated alert information to be displayed. Further, the controlling function 161 causes the display 103 to display the GUI used for having the registration process performed again.

As explained above, the ultrasound diagnosis apparatus according to the first embodiment is configured to maintain a certain level of precision for the registration processes between the ultrasound image and the reference image, by presenting the user with the changes in the degree of similarity and receiving an instruction to perform the re-registration process. In this situation, the ultrasound diagnosis apparatus 1 is also capable of automatically performing the re-registration process, on the basis of the degrees of similarity calculated by the similarity degree calculating function 164. In that situation, the controlling function 161 monitors the degrees of similarity calculated by the similarity degree calculating function 164 and, when the degree of similarity is smaller than or is equal to or smaller than a threshold value, the controlling function 161 generates ultrasound volume data again. Further, the registration function 163 corrects (updates) the correspondence relationship by comparing the re-generated ultrasound volume data with the reference volume data.

In this situation, the threshold value used for judging the degrees of similarity is set on the basis of at least one selected from among: the site scanned by the ultrasound probe 101, the physique of the subject, and the posture of the subject at the time of the acquisition of the three-dimensional medical image data. For example, when a site of which the shape easily changes or a site from which it is difficult to acquire ultrasound image data is subject to a medical examination, the threshold value is set to a smaller value. Conversely, when a site of which the shape does not change easily or a site from which it is easy to acquire ultrasound image data is subject to a medical examination, the threshold value is set to a larger value.

Further, for example, when the subject has a larger physique and the distance (the depth) from the body surface to the target site is longer, the threshold value is set to a smaller value. Conversely, when the distance is shorter, the threshold value is set to a larger value. As another example, when the posture of the subject at the time of acquiring the reference volume data is different from the posture of the subject at the time of acquiring the ultrasound image data, the threshold value is set to a smaller value. Conversely, when the postures are the same for both of the acquisitions, the threshold values is set to a larger value.

As explained above, the controlling function 161 is configured to compare the degrees of similarity calculated by the similarity degree calculating function 164 with the thresh id value, and when at least one of the degrees of similarity is smaller than or is equal to or smaller than the threshold value, the controlling function 161 arranges the ultrasound volume data to be generated. In this situation, the controlling function 161 arranges the three-dimensional ultrasound image data to be generated on the basis of the pieces of two-dimensional ultrasound image data corresponding to the plurality of temporal phases. In other words, by controlling the image generating circuitry 140, the controlling function 161 arranges the ultrasound volume data to be reconstructed by using the plurality of pieces of two-dimensional ultrasound image data. Alternatively, the controlling function 161 may arrange the ultrasound volume data to be generated by having a three-dimensional ultrasound scan performed on the subject.

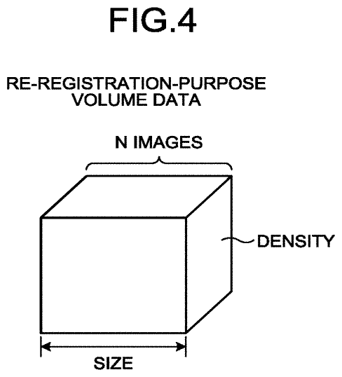

FIG. 4 is a drawing for explaining an example of the ultrasound volume data generating process performed under the control of the controlling function 161 according to the first embodiment. For example, as illustrated in FIG. 4, the controlling function 161 arranges a re-registration-purpose ultrasound volume data to be reconstructed by using two-dimensional ultrasound image data corresponding to N images. In this situation, the quantity “N” of the images in the two-dimensional ultrasound image data used in the re-registration-purpose volume data may arbitrarily be set. For example, the quantity of the images may be set in accordance with the target site, the size of the volume data, or the like.

Further, for example, as illustrated in FIG. 4, the controlling function 161 arranges the ultrasound volume data to be generated so that the re-registration-purpose volume data has a predetermined size. Further, for example, as illustrated in FIG. 4, the controlling function 161 arranges the ultrasound volume data to be generated so that the density of the re-registration-purpose volume data has a predetermined level of density.

When the controlling function 161 has generated the re-registration-purpose volume data in the manner described above, the registration function 163 performs a registration process between the generated re-registration-purpose volume data and the reference volume data. In this situation, the registration process of the registration function 163 may he performed with arbitrary timing or may be performed with timing determined in advance. For example, the registration function 163 may acquire the position information of the ultrasound probe 101 obtained from the position sensor 104 so as to correct (update) the correspondence relationship when the moving amount of the ultrasound probe 101 is smaller than, or is equal to or smaller than, a threshold value. In other words, the registration function 163 corrects (updates) the correspondence relationship between the ultrasound image and the reference image at such a time that involves little movement of the ultrasound probe 101.

For example, when the moving amount the ultrasound probe 101 is large, the user may be searching for a desired site or may be observing the status of the surroundings of the target site, while referencing the ultrasound image. If the correspondence relationship were updated in that situation, there is a possibility that the updating process might impact the displayed images (e.g., the displayed images might not transition from one to another smoothly). To avoid this problem, the registration function 163 updates the correspondence relationship between the ultrasound image and the reference image at such a time that involves little movement of the ultrasound probe 101.

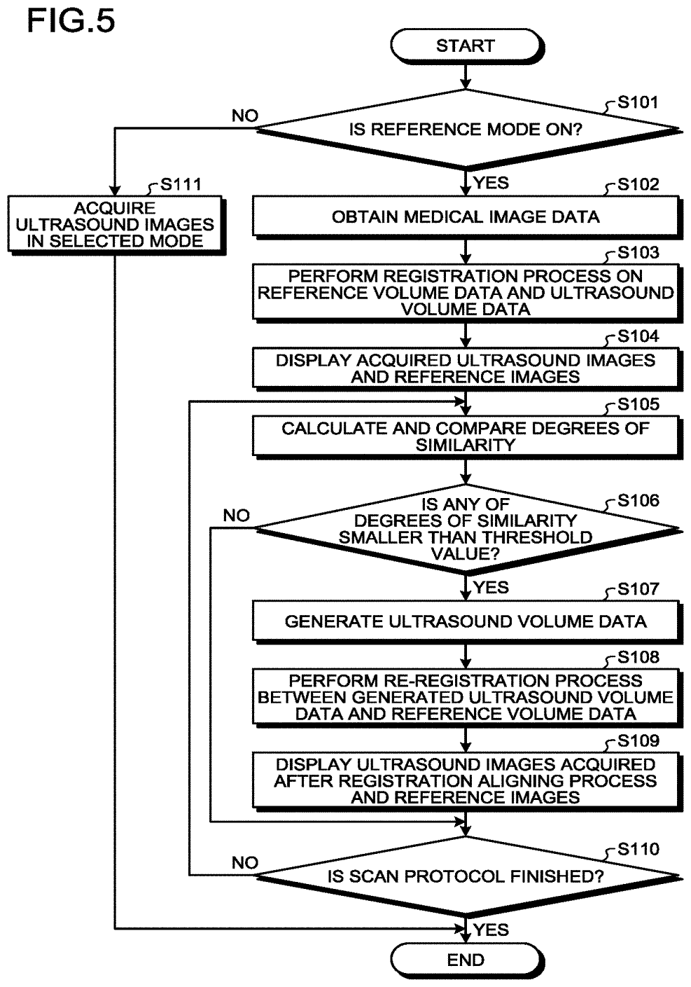

Next, a procedure in a process performed by the ultrasound diagnosis apparatus 1 according to the first embodiment will be explained. FIG. 5 is a flowchart illustrating the processing procedure performed by the ultrasound diagnosis apparatus 1 according to the first embodiment. In the present example, FIG. 5 illustrates the process performed when the re-registration process based on the degrees of similarity is automatically performed.

Steps S101, S102, S104, S106, S107, and S109 through S111 in FIG. 5 are realized, for example, as a result of the processing circuitry 160 reading and executing the program corresponding to the controlling function 161 from the storage 150. Further, steps S103 and S108 are realized, for example, as a result of the processing circuitry 160 reading and executing the program corresponding to the registration function 163 from the storage 150. Further, step S105 is realized, for example, as a result of the processing circuitry 160 reading and executing the program corresponding to the similarity degree calculating function 164 from the storage 150.

In the ultrasound diagnosis apparatus 1 according to the present embodiment, the processing circuitry 160 first judges whether or not the reference mode is on in which the reference images are referenced (step S101). When the reference mode is not on (step S101: No), the processing circuitry 160 acquires ultrasound images in a selected mode (step S111). On the contrary, when the reference mode is on (step S101: Yes), the processing circuitry 160 obtains medical image data (step S102) and performs a registration process between reference volume data and ultrasound volume data (step S103).

Subsequently, the processing circuitry 160 arranges the acquired ultrasound images and corresponding reference images to be displayed (step S104). Further, while the ultrasound images and the reference images are being displayed, the processing circuitry 160 calculates and compares degrees of similarity with the threshold value (step S105) to judge whether any of the degrees of similarity is smaller than the threshold value (step S106). When at least one of the degrees of similarity is smaller than the threshold value (step S106: Yes), the processing circuitry 160 arranges ultrasound volume data to be generated (step S107) and performs a re-registration process between the generated ultrasound volume data and the reference volume data (step S108).

After that, the processing circuitry 160 displays ultrasound images acquired after the registration process and reference images (step S109). Subsequently, the processing circuitry 160 judges whether or not the scan protocol is finished (step S110). When the scan protocol is finished (step S110: Yes), the processing circuitry 160 ends the process. On the contrary, when the scan protocol is not finished (step S110: No), the processing circuitry 160 returns to step S105 where the processing circuitry 160 continues comparing the degrees of similarity. At step S106, when none of the degrees of similarity is smaller than the threshold value (step S106: No), the processing circuitry 160 continues to display the ultrasound images and the reference images and judges whether or not the scan protocol is finished (step S110).

As explained above, according to the first embodiment, the controlling function 161 is configured to arrange the two-dimensional ultrasound scan to be performed on the subject, via the ultrasound probe 101 provided with the position sensor 104. The image generating circuitry 140 is configured to generate the two-dimensional ultrasound image data on the basis of the echo data acquired by the two-dimensional ultrasound scan. On the basis of the position information of the two-dimensional ultrasound image data in the first coordinate space that was specified from the output of the position sensor 104 and the correspondence relationship obtained in advance between the second coordinate space to which the three-dimensional medical image data of the subject belongs and the first coordinate space, the image generating circuitry 140 is configured to reconstruct the two-dimensional medical image data corresponding to the two-dimensional ultrasound image data from the three-dimensional medical image data. Every time the predetermined condition is satisfied, the similarity degree calculating function 164 is configured to calculate the degree of similarity between different one of the sequentially-generated pieces of two-dimensional ultrasound image data and a corresponding one of the sequentially-reconstructed pieces of two-dimensional medical image data. Consequently, the ultrasound diagnosis apparatus 1 according to the first embodiment is configured to observe the changes in the degrees of similarity indicating the levels of precision of the registration processes and thus makes it possible to maintain a certain level of precision for the registration processes and to improve the efficiency of the medical examination.

Further, according to the first embodiment, when at least e of the degrees of similarity is smaller than or is equal to or smaller than the threshold value, the image generating circuitry 140 is configured to generate the three-dimensional ultrasound image data on the basis of the pieces of two-dimensional ultrasound image data corresponding to the plurality of temporal phases. The registration function 163 is configured to update the correspondence relationship by comparing the three-dimensional ultrasound image data with the three-dimensional medical image data. Further, when at least one of the degrees of similarity is smaller than or is equal to or smaller than the threshold value, the controlling function 161 is configured to cause the three-dimensional ultrasound scan to be performed on the subject. The image generating circuitry 140 is configured to generate the three-dimensional ultrasound image data on the basis of the echo data acquired by the three-dimensional ultrasound scan. The registration function 163 is configured to update the correspondence relationship by comparing the three-dimensional ultrasound image data with the three-dimensional medical image data. Consequently, the ultrasound diagnosis apparatus 1 according to the first embodiment is able to update the correspondence relationship as appropriate and makes it possible to automatically maintain a certain level of precision for the registration processes.

Further, according to the first embodiment, the threshold value is set on the basis of at least one selected from among: the site scanned by the ultrasound probe 101, the physique of the subject, and the posture of the subject at the time of the acquisition of the three-dimensional medical image data. Consequently, the ultrasound diagnosis apparatus 1 according to the first embodiment makes it possible to set the threshold value in accordance with the levels of easiness of the registration processes.

Further, according to the first embodiment, the registration function 163 is configured to update the correspondence relationship when the moving amount of the ultrasound probe 101 is smaller than, or equal to or smaller than, the threshold value. Consequently, the ultrasound diagnosis apparatus 1 according to the first embodiment makes it possible to update the correspondence relationship with optimal timing.

Further, according to the first embodiment, the similarity degree calculating function 164 is configured to normalize the degrees of similarity. The controlling function 161 is configured to cause the display 103 to display the normalized degrees of similarity. Consequently, the ultrasound diagnosis apparatus 1 according to the first embodiment makes it possible to display the information with which it is easy to judge the state of the registration processes.

Further, according to the first embodiment, the display information generating function 165 is configured to generate the indicator indicating the degrees of similarity. The controlling function 161 is configured to cause the display 103 to display the indicator. Consequently, the ultrasound diagnosis apparatus 1 according to the first embodiment makes it possible to display the information that enables viewers to judge the state of the registration processes at a glance.

Further, according to the first embodiment, the similarity degree calculating function 164 is configured to calculate the degree of similarity once every predetermined periodic cycle. Consequently, the ultrasound diagnosis apparatus 1 according to the first embodiment makes it possible to reduce processing loads.

Further, according to the first embodiment, the predetermined periodic cycle used for calculating the degrees of similarity is set in accordance with at least one selected from among: the site scanned by the ultrasound probe 101, the physique of the subject, and the posture of the subject during the scan performed by the ultrasound probe 101. Consequently, the ultrasound diagnosis apparatus 1 according to the first embodiment is able to calculate the degrees of similarity with such timing that corresponds to how easily the degrees of similarity can change and therefore makes it possible to reduce processing loads more efficiently.

### Second Embodiment

The first embodiment has thus been explained. It is possible to carry out the present disclosure in various different modes other than those described above in the first embodiment.

In the embodiments described above, the example is explained in which the position sensor 104 obtains the position information of the ultrasound probe 101. However, possible embodiments are not limited to this example. For instance, the position information of the ultrasound probe 101 may be obtained by using a motion sensor, a robot arm, a camera, or the like.

For example, when a motion sensor is used, the motion sensor is attached to the ultrasound probe 101, and an initial state of the ultrasound probe 101 is set. In one example, the obtaining function 162 is configured to set predetermined state of t ultrasound probe 101 to which the motion sensor Is attached, as the initial state. Further, the obtaining function 162 is configured to obtain displacement amounts in the position and the orientation of the ultrasound probe 101, on the basis of differences between a plurality of pieces of motion information that are chronologically obtained as the ultrasound probe 101 moves and the initial state.

For example, the registration function 163 brings the site included in the ultrasound image data acquired in the initial state and the corresponding site in the reference volume data into substantially the same position as each other. With this arrangement, the registration function 163 determines a correspondence relationship between the ultrasound image data and the reference volume data.

In another example, when a robot arm is used, the ultrasound probe 101 is held by the robot arm, so that a scan is performed on the subject as a result of the robot arm moving the ultrasound probe 101. In that situation, the obtaining function 162 sets a predetermined position of the ultrasound probe 101 held by the robot arm as an initial position. Further, the obtaining function 162 obtains a moving amount from the initial position with respect to the robot arm holding the ultrasound probe 101, as displacement amounts in the position and the orientation of the ultrasound probe 101.

For example, the registration function 163 brings the site included in the ultrasound image data acquired in the initial position and the corresponding site in the reference volume data into substantially the same position as each other. With this arrangement, the registration function 163 determines a correspondence relationship between the ultrasound image data and the reference volume data.

In yet another example, when a camera is used, a marker or the like is attached to the ultrasound probe 101, so as to obtain displacement amounts in the position and the orientation of the ultrasound probe on the basis of changes in the position of the marker imaged by the camera. In that situation, the obtaining function 162 sets the position of the marker in a predetermined state of the ultrasound probe 101 as an initial position. Further, the obtaining function 162 obtains the displacement amounts in the position and the orientation of the ultrasound probe 101, on the basis of differences between positions of the marker that are chronologically obtained as the ultrasound probe 101 moves and the initial position.

For example, the registration function 163 brings the site included in the ultrasound image data acquired in the initial position and the corresponding site in the reference volume data into substantially the same position as each other. With this arrangement, the registration function 163 determines a correspondence relationship between the ultrasound image data and the reference volume data.

The constituent elements of the apparatuses and the devices illustrated in the drawings in the above embodiments are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analysed and executed by the CPU or may be realized as hardware using wired logic.

Further, with regard to the processes explained in the embodiments above, it is acceptable to manually perform all or a part of the processes described as being performed automatically. Conversely, by using a method that is publicly known, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

Further, the medical information processing methods explained in the above embodiments may be realized by causing a computer such as a personal computer or a workstation to execute a medical information processing program prepared in advance. The medical information processing methods may be distributed via a network such as the Internet. Further, the medical information processing methods may be recorded on a computer-readable recording medium such as a hard disk, a flexible disk (CD), a CD-ROM, an MO disk, or a DVD, so as to be executed as being read from the recording medium by a computer.

According to at least one aspect of the embodiments described above, it is possible to maintain a certain level of precision for the registration processes and to therefore improve efficiency of the medical examinations.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
processing circuitry configured to
perform a two-dimensional ultrasound scan on a subject via an ultrasound probe;
generate two-dimensional ultrasound image data on a basis of echo data acquired by the two-dimensional ultrasound scan;
reconstruct two-dimensional medical image data from three-dimensional medical image data of the subject, on a basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space; and
calculate a degree of similarity between the two-dimensional ultrasound image data and the two-dimensional medical image data every time a condition is satisfied.

2. The ultrasound diagnosis apparatus according to claim 1, wherein the processing circuitry is configured to
when at least one of the degrees of similarity is smaller than, or equal to or smaller than, a threshold value, generate three-dimensional ultrasound image data on a basis of the two-dimensional ultrasound image data corresponding to a plurality of temporal phases, and
correct the correspondence relationship by comparing the three-dimensional ultrasound image data with the three-dimensional medical image data.

3. The ultrasound diagnosis apparatus according to claim 1, wherein the processing circuitry is configured to
when at least one of the degrees of similarity is smaller than, or equal to or smaller than, a threshold value, cause a three-dimensional ultrasound scan to be performed on the subject,
generate three-dimensional ultrasound image data on a basis of echo data acquired by the three-dimensional ultrasound scan, and
correct the correspondence relationship by comparing the three-dimensional ultrasound image data with the three-dimensional medical image data.

4. The ultrasound diagnosis apparatus according to claim 3, wherein the threshold value is set on a basis of at least one selected from among: a site scanned by the ultrasound probe, a physique of the subject, and a posture of the subject at a time of acquisition of the three-dimensional medical image data.

5. The ultrasound diagnosis apparatus according to claim 3, wherein the processing circuitry is configured to execute the correction of the correspondence relationship at a time when a moving amount of the ultrasound probe is smaller than, or equal to or smaller than, a threshold value.

6. The ultrasound diagnosis apparatus according claim 1, wherein the processing circuitry is configured to normalize the degrees of similarity and cause a display to display the normalized degrees of similarity.

7. The ultrasound diagnosis apparatus according to claim 1, wherein the processing circuitry is configured to generate an indicator indicating the degrees of similarity and cause a display to display the indicator.

8. The ultrasound diagnosis apparatus according to claim 1, wherein the processing circuitry is configured to calculate the degree of similarity once every predetermined periodic cycle.

9. The ultrasound diagnosis apparatus according to claim 8, wherein the predetermined periodic cycle is defined by one selected from among: a predetermined time interval, a predetermined frame interval, and every time the ultrasound probe has moved a predetermined distance.

10. The ultrasound diagnosis apparatus according to claim 8, wherein the predetermined periodic cycle is set in accordance with at least one selected from among: a site scanned by the ultrasound probe, a physique of the subject, and a posture of the subject during the scan performed by the ultrasound probe.

11. An ultrasound diagnosis apparatus comprising:
processing circuitry configured to
perform a three-dimensional ultrasound scan on a subject via an ultrasound probe;
generate three-dimensional ultrasound image data on a basis of echo data acquired by the three-dimensional ultrasound scan;
reconstruct two-dimensional ultrasound image data from the three-dimensional ultrasound image data; and reconstruct two-dimensional medical image data from three-dimensional medical image data of the subject on a basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space; and
calculate degree of similarity between the two-dimensional ultrasound image data and the two-dimensional medical image data every time a condition is satisfied.

12. The ultrasound diagnosis apparatus according to claim 11, wherein the processing circuitry is configured to
when at least one of the degrees of similarity is smaller than, or equal to or smaller than, a threshold value, generate three-dimensional ultrasound image data on a basis of the two-dimensional ultrasound image data corresponding to a plurality of temporal phases, and
correct the correspondence relationship by comparing the three-dimensional ultrasound image data with the three-dimensional medical image data.

13. The ultrasound diagnosis apparatus according to claim 11, wherein the processing circuitry is configured to
when at least one of the degrees of similarity is smaller than, or equal to or smaller than, a threshold value, cause a three-dimensional ultrasound scan to be performed on the subject,
generate three-dimensional ultrasound image data on a basis of echo data acquired by the three-dimensional ultrasound scan, and
correct the correspondence relationship by comparing the three-dimensional ultrasound image data with the three-dimensional medical image data.

14. The ultrasound diagnosis apparatus according to claim 13, wherein the threshold value is set on a basis of at least one selected from among: a site scanned by the ultrasound probe, a physique of the subject, and a posture of the subject at a time of acquisition of the three-dimensional medical image data.

15. The ultrasound diagnosis apparatus according to claim 13, wherein the processing circuitry is configured to execute the correction of the correspondence relationship at a time when a moving amount of the ultrasound probe is smaller than, or equal to or smaller than, a threshold value.

16. The ultrasound diagnosis apparatus according to claim 11, wherein the processing circuitry is configured to normalize the degrees of similarity and cause a display to display the normalized degrees of similarity.

17. The ultrasound diagnosis apparatus according to claim 11, wherein the processing circuitry is configured to generate an indicator indicating the degrees of similarity and cause a display to display the indicator.

18. The ultrasound diagnosis apparatus according to claim 11, wherein the processing circuitry is configured to calculate the degree of similarity once every predetermined periodic cycle.

19. The ultrasound diagnosis apparatus according to claim 18, wherein the predetermined periodic cycle is defined by one selected from among: a predetermined time interval, a predetermined frame interval, and every time the ultrasound probe has moved a predetermined distance.

20. The ultrasound diagnosis apparatus according to claim 18, wherein the predetermined periodic cycle is set in accordance with at least one selected from among: a site scanned by the ultrasound probe, a physique of the subject, and a posture of the subject during the scan performed by the ultrasound probe.

21. A medical information processing method comprising:
performing a two-dimensional ultrasound scan on a subject via an ultrasound probe;
generating two-dimensional ultrasound image data on a basis of echo data acquired by the two-dimensional ultrasound scan;
reconstructing two-dimensional medical image data from three-dimensional medical image data of the subject, on a basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space; and
calculating a degree of similarity between the two-dimensional ultrasound image data and the two-dimensional medical image data every time a condition is satisfied.

22. A medical information processing method comprising:
performing a three-dimensional ultrasound scan on a subject via an ultrasound probe;
generating three-dimensional ultrasound image data on a basis of echo data acquired by the three-dimensional ultrasound scan;
reconstructing two-dimensional ultrasound image data from the three-dimensional ultrasound image data; and reconstructing two-dimensional medical image data from three-dimensional medical image data of the subject on a basis of position information of the two-dimensional ultrasound image data in a first coordinate space specified from detected position information of the ultrasound probe and a correspondence relationship obtained in advance between a second coordinate space to which the three-dimensional medical image data belongs and the first coordinate space; and
calculating a degree of similarity between the two-dimensional ultrasound image data and the two-dimensional medical image data every time a condition is satisfied.
